(19) United States
(12) Patent Application Publication
(10) Pub. No.: US 2004/0044217 A1
(43) Pub. Date: Mar. 4, 2004

(54) Process to produce halo-4-phenoxyquinolines

(76) Inventors: Karl Leopold Krumel, Midland, MI (US); Ruth Cezar Reed, Saginaw, MI (US); Thomas A. Olmstead, Saginaw, MI (US); Gary Alan Roth, Midland, MI (US); Ian Robert King, King's Lynn (GB); Donald Neil Brattesani, Oakland, CA (US); Kent Douglas Campbell, Concord, CA (US); John Davies, Midland, MI (US)

Correspondence Address: DOW AGROSCIENCES LLC, 9330 ZIONSVILLE RD, INDIANAPOLIS, IN, 46268, US

(21) Appl. No.: 10/363,025

(22) PCT Filed: Sep. 7, 2001
(86) PCT No.: PCT/US01/27689

(51) Int. Cl. C07D 213/08; C07D 213/14; C07C 229/40

(52) U.S. Cl. 546/250; 560/38

(57) ABSTRACT

The present invention relates to a process for the preparation of halo-4-phenoxyquinolines.

### PRIORITY

This application claims priority from U.S. provisional application serial No. 60/231,719 which was filed on Sep. 8, 2000.

### FIELD OF THE INVENTION

This invention is related to the field of processes to produce halo-4-phenoxyquinolines.

### BACKGROUND OF THE INVENTION

Our history is riddled with outbreaks of fungal diseases that have caused widespread human suffering. One need look no further than the Irish potato famine of the 1850's, where an estimated 1,000,000 people died, to see the effects of a fungal disease.

Fungicides are compounds, of natural or synthetic origin, which act to protect plants against damage caused by fungi. Current methods of agriculture rely heavily on the use of fungicides. In fact, some crops cannot be grown usefully without the use of fungicides. Using fungicides allows a grower to increase the yield of the crop and consequently, increase the value of-the-crop. In most situations, the increase in value of the crop is worth at least three times the cost of the use of the fungicide.

However, research is being conducted to lower the cost of producing fungicides, thereby lowering the costs associated with using fungicides. In light of the above, the inventors provide this invention.

### SUMMARY OF THE INVENTION

It is an object of this invention to provide a process to produce halo-4-phenoxyquinolines. In accordance with this invention a process is provided, said process comprising:

(1) reacting a compound of formula one with a compound of formula two to produce a compound of formula three;

(2) reacting said compound of formula three to produce a compound of formula four;

(3) reacting said compound of formula four with a compound of formula five to produce a compound of formula six;

(4) reacting said compound of formula six with a compound of formula seven to produce a compound of formula eight; and

(5) reacting said compound of formula eight with a compound of formula nine to produce a compound of formula ten.

### DETAILED DESCRIPTION OF THE INVENTION

The first step in this process is reacting a compound of formula one with a compound of formula two to produce a compound of formula three, as shown in the reaction scheme.

In the compound of formula one (also referred to herein as “Compound One” or “Aniline Compound”) R¹ and R³ are independently halo and R² and R⁴ are H; or R³ is halo, R¹ is halo or H, and R² and R⁴ are H; or R⁴ is halo and R¹ to R³ are H. Currently, it is preferred when 3,5-dichloroaniline (also referred to herein as “DCA”) is used as Compound One.

In the compound of formula two (also referred to herein as “Compound Two” or “Alkoxymethylene Malonate Dialkyl Ester Compound”) R⁵ is a (C₁-C₄) alkyl and R⁶ and R⁷ are independently a (C₁-C₄) alkyl. Currently, it is preferred when diethyl ethoxymethylenemalonate (also referred to herein as “EMME”) is used as Compound Two.

The compound of formula three is also referred to herein as “Compound Three” or “Aniline-Alkoxymethylene Malonate Dialkyl Ester-Product Compound”. The R groups are the same as defined in Compound One and Compound Two.

The reaction is conducted by subjecting a reaction mixture comprising Compound One and Compound Two to a temperature from about 50 to about 210° C., preferably from about 160 to about 210° C., and a pressure from about 5 psia to about 50 psia, for a time period sufficient to drive the reaction to completion, usually about 0.5-3 hours. Currently, it is preferred when a slight molar excess of the Compound Two to Compound One is used.

This step of the process can be conducted in the presence of a solvent, however, a solvent is not required. Any solvent that does not react with Compound One or Compound Two under the reaction conditions of this step is useful. Preferably this solvent has a boiling point above the reaction temperature when subjected to the reaction conditions. Solvents such as, for example, sulfolane, tetraglyme, polyethers, long-chain (C₁₀₋₄₀) alkylaromatics; C₁₋₆ alkylated naphthalene, naphthalene, diesel or fuel oil can be suitable. Currently, it is preferred if a hydrocarbyl aromatic compound, preferably a (C₁-C₃₀) alkyl substituted hydrocarbyl aromatic compound, is used. Currently, it is more preferred to use dodecylbenzene, however, in practical terms, it is probably less-expensive to use a composition that contains mixtures of (C₁-C₃₀)alkyl-substituted aryl compounds.

The second step in this process is reacting the compound of formula three to produce a compound of formula four, as shown in the reaction scheme.

In the compound of formula four (also referred to herein as “Compound Four” or “Alkoxycarbonyl Quinoline Compound”) R⁸ is a (C₁-C₄) alkyl.

The reaction is conducted by subjecting a reaction mixture comprising Compound Three to a temperature from about 200-270° C., preferably from about 220-250° C., and a pressure from about 5 psia to about 50 psia, for a time period sufficient to drive the reaction to completion, usually about 2-20 hours.

This step of the process is preferably conducted in the presence of a solvent. The solvents discussed above in step one can be used.

Optionally, this step can include recovering said Compound Four before proceeding to step three of the process. This recovery can be accomplished by any means which isolates Compound Four such as, for example, as discussed in U.S. Pat. No. 5,973,153 (hereby incorporated by reference). If Compound Four is isolated it can optionally be converted to an acid by exchanging the R⁸ for hydrogen (—H).

The third step in this process is reacting the compound of formula four with a compound of formula five to produce a compound of formula six, as shown in the reaction scheme.

The compound of formula five (also referred to herein as “Compound Five” or “Hydroxy Compound”) is any compound that contains a hydroxy group (—OH) and that interacts with the —C(═O)—O—R⁸ group on Compound Four to produce a compound of formula six (also referred to herein as “Compound Six” or “Hydroxy Quinoline Compound”). Currently, H₂O is the preferred compound to use as Compound Five.

The reaction is conducted by subjecting a reaction mixture comprising Compound Four and Compound Five to a temperature from about 180-270° C., preferably from about 200-230° C., and a pressure from about 5 psia to about 50 psia, for a time period sufficient to drive the reaction to completion, usually about 2-20 hours.

This step of the process is preferably conducted in the presence of a solvent. The solvents discussed above in step one can be used. Currently, it is preferred if a polar hydrocarbylhetero compound, preferably a (C₁-C₁₀) polar hydrocarbylhetero compound, is used. Currently, it is more preferred to use sulfolane. It is preferred to use a weight ratio of 5:1 to 20:1 solvent:Compound Four.

If desired it is possible to isolate Compound Six before reacting it with Compound Seven in a solvent. Additionally, if desired it is possible to dry Compound Six before reacting it with Compound Seven in a solvent.

The fourth step in the this process is reacting the compound of formula six with a compound of formula seven to produce a compound of formula eight, as shown in the reaction scheme.

In the compound of formula seven (also referred-to herein as “Compound-Seven” or “Halo Compound”) is any compound that contains a halo group (—F, Cl, Br, or I) and that interacts with the —OH group on Compound Six to produce a compound of formula Eight (also referred to herein as “Compound Eight” or “Halo Quinoline Compound”). Currently, SOCl₂ (aka thionyl chloride) is the preferred compound to use as Compound Seven.

The reaction is conducted by subjecting a reaction mixture comprising Compound Six and Compound Seven to a temperature from about 50-100° C., preferably from about 60-90° C., and a pressure from about 5 psia to about 50 psia, for a time period sufficient to drive the reaction to completion, usually about 0.5-5 hours.

This step of the process is preferably conducted in the presence of a solvent. The solvents discussed above in step one can be used. Currently, it is preferred when a polar hydrocarbylhetero compound is used. It is also preferred to use a catalyst to increase to rate of reaction. Currently, N,N-dimethylformamide and N,N-diethylformamide are preferred catalysts.

Currently, it is preferred to remove any of Compound Seven or other unwanted side products before proceeding to step five of the process.

The fifth step in this process is reacting the compound of formula eight with a compound of formula nine to produce a compound of formula ten, as shown in the reaction scheme.

In the compound of formula nine (also referred to herein as “Compound Nine” or “Aryloxy Compound”) R⁹ to R¹³ are independently H, CN, NO₂, OH, halo, (C₁-C4)alkyl, (C₂-C₄)alkanoyl, halo(C₁-C₇)alkyl, hydroxy(C₁-C₇)alkyl, (C₁-C7)alkoxy, halo(C₁-C₇)alkoxy, (C₁-C₇)alkylthio, halo(C₁-C₇)alkylthio, phenyl, substituted phenyl, phenoxy, substituted phenoxy, phenylthio, substituted phenylthio, phenyl(C₁-C₄)alkyl, substituted phenyl(C₁-C₄)alkyl, benzoyl, SiR²OR²¹R²², or OSiR²OR²¹R²² where R²⁰, R²¹, and R²² are H, a (C₁-C₆)alkyl group, phenyl, or substituted phenyl, provided that at least one of R²⁰, R²¹, and R²² is other than H, or R¹¹ and R¹² or R¹² and R¹³ combine to form a carbocyclic ring, and provided that unless all of R⁹ to R¹³ are H or F, then at least two of R⁹ to R¹³ are H. Wherein the foregoing definitions, the term substituted phenyl refers to phenyl substituted with up to three groups selected from halo, (C₁-C₁₀)alkyl, halo(C₁-C₇)alkyl, hydroxy(C₁-C₇)alkyl, (C₁-C₇)alkoxy, halo(C₁-C₇)alkoxy, phenoxy, phenyl, NO₂, OH, CN, (C₁-C₄)alkanoyloxy, or benzyloxy. The term alkyl refers to linear, branched, or cyclic alkyl. The term halo refers to fluoro, chloro, bromo, or iodo. The term substituted phenoxy refers to a phenoxy group substituted with up to three groups selected from halo, (C₁-C₁₀)alkyl, halo(C₁-C₇)alkyl, hydroxy-(C₁-C₇)alkyl, (C₁-C₇)alkoxy, halo(C₁-C₇)alkoxy, phenoxy, phenyl, NO₂, OH, CN, (C₁-C₄)alkanoyloxy, or benzyloxy. The term substituted phenylthio refers to a phenylthio group substituted with up to three groups selected from halo, (C₁-C₁₀)alkyl, halo(C₁-C₇)alkyl, hydroxy(C₁-C₇)alkyl, (C₁-C₇)alkoxy, halo(C₁-C₇)alkoxy, phenoxy, phenyl, NO₂, OH, CN, (C₁-C₄)alkanoyloxy, or benzyloxy. Currently, it is preferred to use parafluorophenol as Compound Nine.

The reaction is conducted by subjecting a reaction mixture comprising Compound Eight and Compound Nine to a temperature from about 40-70° C., preferably from about 55-65° C., and a pressure from about 0.5 psia to about 50 psia, for a time period sufficient to drive the reaction to completion, usually about 0.5-20 hours.

The step of the process is preferably conducted in the presence of a solvent. The solvents discussed above in step one can be used. Currently, it is preferred when a polar hydrocarbylhetero compound is used.

Currently, it is preferred to use a base during the reaction. Any base that promotes this coupling reaction can be used. Currently it is preferred when the base contains a —OH group. Suitable bases are triethylamine, sodium carbonate, potassium carbonate, sodium hydroxide, and potassium hydroxide. Currently, potassium hydroxide is preferred.

Optionally, step five can include recovering said compound of formula ten. This recovery can be accomplished by any means that isolates Compound Ten such as, for example, as discussed in U.S. Pat. No. 5,973,153.

Currently, it is preferred to use dodecylbenzene as a solvent in steps one and two, and sulfolane as a solvent in steps 3-5. Additionally, it is preferred to isolate Compound Four before proceeding to step three.

### EXAMPLES

These examples are provided to further illustrated the invention to one skilled in the art. They are not meant to be construed as limiting the invention.

### Example One

### Preparation of fluoro-4-phenoxy-5,7-dichloroquinoline

The compounds 3,5-dichloroaniline (DCA, 162.7 g, 1.0 mole) and diethyl ethoxy-methylenemalonate (EMME, 221.1 g, 1.02 mole) were mixed into 1.135 kg of recycle dodecylbenzene and 83.3 g of fresh dodecylbenzene. The reaction was carried out in a stainless steel 3-liter, 3-neck round bottom flask equipped with a mechanical agitator and Dean Stark distillation apparatus. The reaction mixture was agitated and nitrogen purged below the liquid level at a rate of 200 cc/min. The reaction mixture was heated to 180° C. over a 60 minute period, then heated to 200° C. for 30 minutes, collecting about one equivalent of ethanol overhead in a Dean Stark apparatus. The mixture was then cooled to room temperature and left overnight under a nitrogen purge.

The reaction mixture was then heated to 240° C. for about 7 hours while purging nitrogen below the liquid level at 180 cc/min and agitating at about 450 RPM. Approximately one equivalent of ethanol was collected overhead during this period. The mixture was cooled to 130° C. and filtered hot using a Buchner funnel with Whatman number 1 paper. The cake was washed with 150 ml of fresh dodecylbenzene and the total filtrate (1066.3 g) removed for recycle. The cake was then washed with two 377 g (500 ml) of recycle ethanol followed by one washed with 377 g of distilled ethanol. The cake was then washed with 500 ml of hot (50° C.) water. This yielded a 439.7 g water wet-cake of Compound Four.

The wet cake, 431.4 g (after sample removal), was then added to 1.8 kg (about 1.43 Liter) of recycle sulfolane in a 5-Liter glass reactor. The residual water (from the wet cake) was distilled at atmospheric pressure from the solution at 90° to 215° C., recovering about 130.9 g of water (some water lost with the vent gases). Care was taken not to super-heat the mixture and nitrogen was purged through the reactor at about 200 cc/min. After reaching a temperature of 200° C. the nitrogen purge was removed and the mixture slowly heated to 220° C. Water was then added at a rate of 6 g/hour over a five hour period, collecting 56 g of ethanol/water in a Dean Stark trap, while maintaining a temperature of 219° C. Heating continued at 220° C. for an additional 2 hours to insure complete reaction. The reaction mixture was then cooled overnight. The reaction mixture was then heated to 120° C. and the residual water removed at reduced pressure (25 mm Hg) for one hour. The reaction mixture was then treated with 3.71 g (0.05 mole) of dimethylformamide and 155.7 g (1.31 mole) of thionyl chloride at a temperature of 75° C. for two hours. The excess thionyl chloride was then removed at reduced pressure (25 mm Hg) at 90° C. for 2 hours recovering 25.7 g of thionyl chloride and sulfur dioxide in the initial trap. The contents were then kept under a nitrogen pad at room temperature over a 3-day weekend (trichloroquinoline compound mixture).

A 5 liter stainless steel reactor was charged with 102.1 g (0.91 mole) of parafluorophenol and 300.7 g (2.41 mole) of 45% potassium hydroxide and mixed at room temperature. This trichloroquinoline solution (in the glass reactor) was mixed and heated to 50° C. to increase the mobility of the slurry and to break-up any solids that formed. The trichloroquinoline mixture was then added to the solution in the 5-liter stainless steel reactor, noting an exotherm going from 21° C. to 51° C. The reactor was slurried with 81.3 g of fresh sulfolane and the mixture used to flush solids that were stuck to the funnel used to add the slurry. The reaction mixture was then heated to 66° C. for four hours at an agitation rate of 600 RPM. After the 4 hours of heating at 66° C., a water addition pump was set-up and water added to the reaction mixture using an accelerated 4-tier rate. For 40 minutes water was added at a rate of 5 ml/min. Then for the next 20 minutes the rate was increased to 10 ml/min. The rate was doubled to 20 ml/min for the next 10 minutes and finally 40 ml/min (15 minutes) until 1200 ml of water had been added to the reactor. The mixture was stirred and cooled to 50° C. prior to emptying the reactor in a 4-liter beaker. The exit valve of the reactor was flushed with 227 ml of fresh water, which was added to the quenched reaction mixture. The reaction mixture was then filtered and washed with 500 ml of hot (50° C.) water recovering 3665.3 g of filtrate solution for sulfolane recovery. The wet cake was then washed a second time with 500 ml of hot water recovering 342.8 g of water-wet cake which contained Compound Ten.

### Example Two

### Preparation of fluoro-4-phenoxy-5,7-dichloroquinoline

This example illustrates steps 3-5 of the process. 7018 kg of wet (˜3 percent by weight water) sulfolane was loaded into a reactor followed by loading 906 kg of 3,5-dichloro-3-carboxy-4-hydroxyquinoline to form a wet slurry. The slurry was heated to 220° C. at atmospheric pressure to remove the majority of the water and held at that temperature for 5 hours to produce 3,5-dichloro-4-hydroxyquinoline. This mixture was cooled to 105° C. and full vacuum was applied for 3 hours to remove water. The mixture was then transferred to another reactor using an additional 750 kg of sulfolane to flush the transfer lines, and further cooled to 70° C. then 522 kg of thionyl chloride and 13.4 kg N,N-dimethylformamide were added and the 3,5-dichlorohydroxyquinoline was reacted at 75° C. for 2 hours to produce 4,5,7-trichloroquinoline. The mixture was then heated to 95° C. with full vacuum for 3 hours to remove SO₂ and SOCl₂, then cooled to 60° C. prior to transfer. The next reactor was preloaded with 1077 kg of 45% KOH aqueous solution and 373 kg parafluorophenol before receiving the 4,5,7-trichloroquinoline mixture and receiving an additional 600 kg of sulfolane to flush the lines. This mixture was then reacted at 60° C. for 12 hours without water removal to make fluoro-4-phenoxy-5,7-dichloroquinoline. 5760 kg of water was then added over 4 hours and then cooled to 40° C. to form the product crystals. The mixture was then fed to two centrifuges to recover the product. The solids were washed with water.

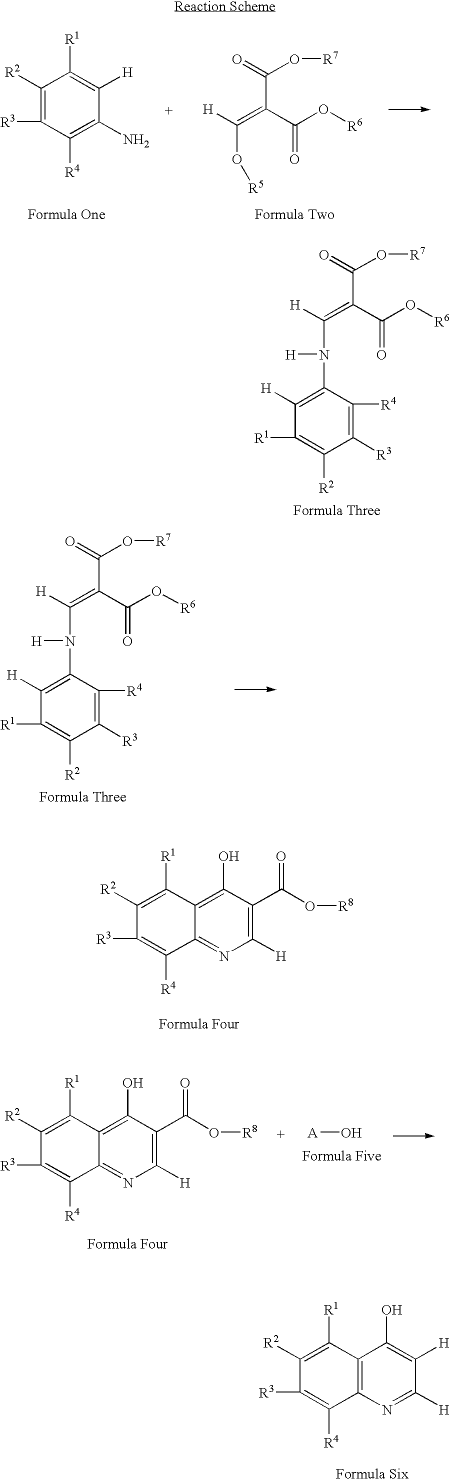

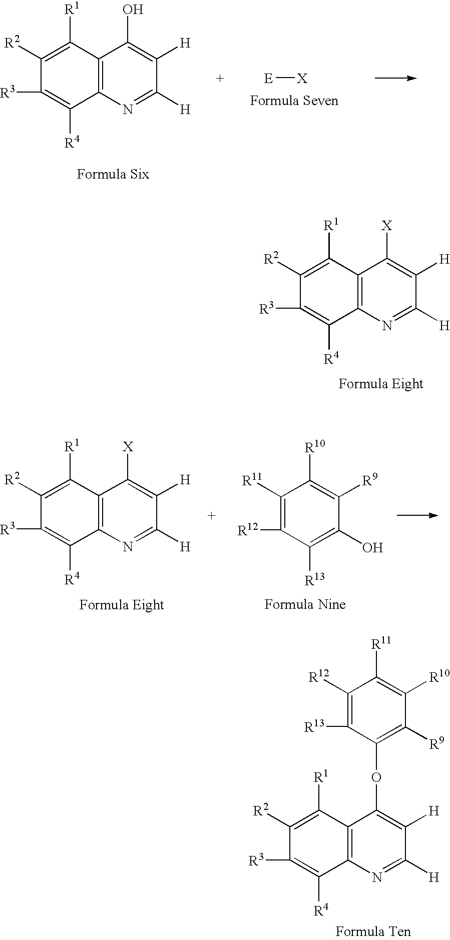

What is claimed is:

1. A process comprising:
(1) reacting a compound of formula one with a compound of formula two to produce a compound of formula three 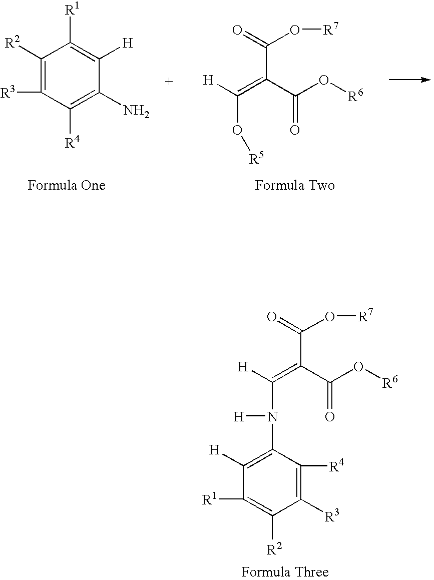
wherein R¹ and R³ are independently halo and R² and R⁴ are H; or R³ is halo, R¹ is halo or H, and R² and R⁴ are H; or R⁴ is halo and R¹ to R³ are H; R⁵ is a (C₁-C₄) alkyl and R⁶ and R⁷ are independently a (C₁-C₄) alkyl;
(2) reacting said compound of formula three to produce a compound of formula four 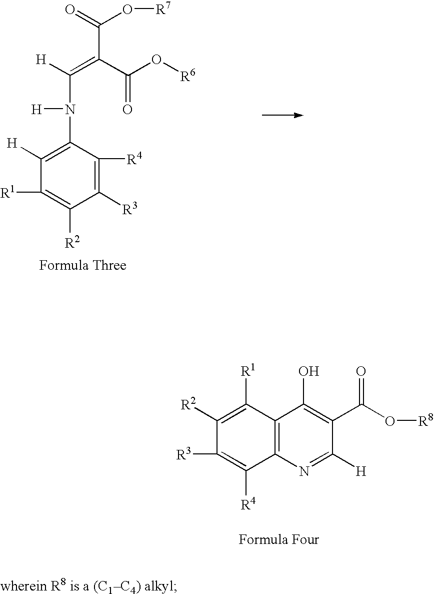
(3) reacting said compound of formula four with a compound of formula five to produce a compound of formula six 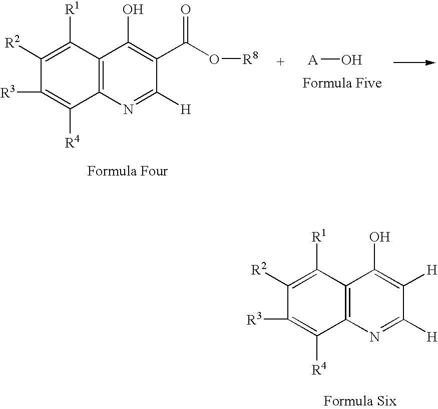
wherein A-OH is is any compound that contains a hydroxy group (OH) and that interacts with the —C(═O)—O—R⁸ group on Compound Four to produce a compound of formula six;
(4) reacting said compound of formula six with a compound of formula seven to produce a compound of formula eight 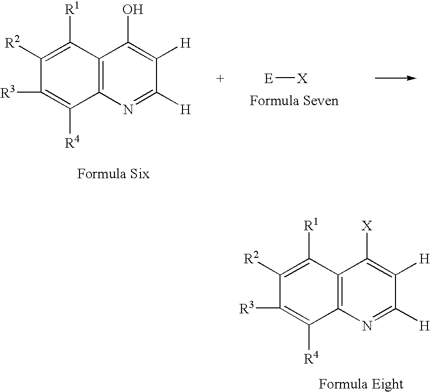
wherein E-X is any compound that contains a halo group (—F, Cl, Br, or 1) and that interacts with the —OH group on Compound Six to produce a compound of Formula Eight; and
(5) reacting said compound of formula eight with a compound of formula nine to produce a compound of formula ten 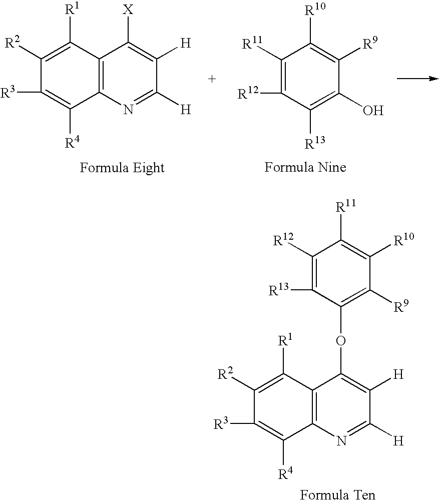
wherein R⁹ to R¹³ are independently H, CN, NO₂, OH, halo, (C₁-C₄)alkyl, (C₂-C₄)alkanoyl, halo(C₁-C₇)alkyl, hydroxy(C₁-C₇)alkyl, (C₁-C₇)alkoxy, halo(C₁-C₇)alkoxy, (C₁-C₇)alkylthio, halo(C₁-C₇)alkylthio, phenyl, substituted phenyl, phenoxy, substituted phenoxy, phenylthio, substituted phenylthio, phenyl(C₁-C₄)alkyl, substituted phenyl(C₁-C₄)alkyl, benzoyl, SiR²⁰R²¹R²², or OSiR²⁰R²¹R²² where R²⁰, R²¹, and R²² are H, a (C₁-C₆)alkyl group, phenyl, or substituted phenyl, provided that at least one of R²⁰, R²¹, and R²² is other than H, or R¹¹ and R¹² or R¹² and R¹³ combine to form a carbocyclic ring, and provided that unless all of R⁹ to R¹³ are H or F, then at least two of R⁹ to R¹³ are H.

2. A process according to claim 1 wherein at least one of steps (1)-(5) is conducted in a solvent.

3. A process according to claim 2 wherein in said solvent is selected from the group consisting of sulfolane, tetraglyme, polyethers, long-chain (C₁₀₋₄₀) alkylaromatics, C₁₋₆ alkylated naphthalene, naphthalene, diesel, fuel oil, and mixtures thereof.

4. A process according to claim 3 wherein said compound of formula one is 3,5-dichloroaniline and said compound of formula two is diethyl ethoxymethylenemalonate.

5. A process-according to claim 4 wherein dodecylbenzene is used as said solvent in steps (1) and (2) and sulfolane is used as said solvent in steps (3)-(5).
